(19) United States
(12) Patent Application Publication
(10) Pub. No.: US 2003/0155431 A1
(43) Pub. Date: Aug. 21, 2003

(54) Fertilizer dispensing device

(76) Inventor: Chih-Chun Hsieh, Miao-Li Hsien (TW)

Correspondence Address: FROMMER LAWRENCE & HAUG, 745 FIFTH AVENUE- 10TH FL., NEW YORK, NY, 10151, US

(21) Appl. No.: 10/127,623

(22) Filed: Apr. 22, 2002

(30) Foreign Application Priority Data
Feb. 7, 2002 (TW) 0912015010

(51) Int. Cl. A61L 9/04

(52) U.S. Cl. 239/34

(57) ABSTRACT

A fertilizer dispensing device includes a tubular body which confines a chamber for receiving granular fertilizers having a first dimension, and which is adapted to be inserted into and buried by soil. The fertilizer dispensing device has a dispensing port formed through the tubular body. The dispensing port has a second dimension smaller than the first dimension so that the granular fertilizers could be carried through the port and into the soil when dissolved.

### CROSS-REFERENCE TO RELATED APPLICATION

This application claims priority of Taiwan Application No. 091201510, filed on Feb. 7, 2002.

### BACKGROUND OF THE INVENTION

1. Field of the Invention

This invention relates to a fertilizer dispensing device, more particularly to a fertilizer dispensing device which is permeable and which can contain the odor of fertilizers.

2. Description of the Related Art

A conventional method for growing plants generally relies on the use of flowerpots to first grow seedlings to an acceptable level and on the subsequent transfer of the plants to desired planting locations. Conventional plants for decoration also use flowerpots for growing. Therefore, the plants not only require planting in highly nutritious soil, but also fertilizers in order to provide adequate nutrition for the soil during the plants' early stage of growth. FIG. 1 shows a conventional flowerpot 1. Typically, fertilizers 2 are spread directly on top of the soil and, in combination with watering, the fertilizers permeate into the soil and are absorbed by the roots of the plants. The disadvantage of the conventional flowerpot 1 lies in that, when the flowerpot 1 is used for decorative purposes, the exposed fertilizers 2 would result in the spreading of the odor of the fertilizers 2 and attracting of insects, which impose negative influences on human bodies and the environment.

### SUMMARY OF THE INVENTION

The object of the present invention is to provide a fertilizer dispensing device adapted to be inserted into and buried by soil so as to enhance nutrition absorption by the roots of the plants, contain the spread of the odor of fertilizers, and prevent the attraction of insects.

According to this invention, the fertilizer dispensing device includes a tubular body adapted to be inserted into and buried by soil. The tubular body has a bottom wall with a periphery, and a tubular wall extending from the periphery of the bottom wall in a longitudinal direction and cooperating with the bottom wall to confine a chamber. The tubular body further has an inner wall surface confronting the chamber, and an outer wall surface opposite to the inner wall surface and distal to the chamber. A smallest one of granular fertilizers has a first dimension. An upper tubular end extends from the tubular wall in the longitudinal direction to define an access opening to permit filling of the chamber with the granular fertilizers. A closure member is disposed to close the access opening. A dispensing port is formed in the inner wall surface, extends through the outer wall surface, and has a second dimension smaller than the first dimension. The granular fertilizers are retained in the chamber until the granular fertilizers are dissolved so as to be carried through the port and into the soil.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 illustrates a conventional method for fertilizing a plant;

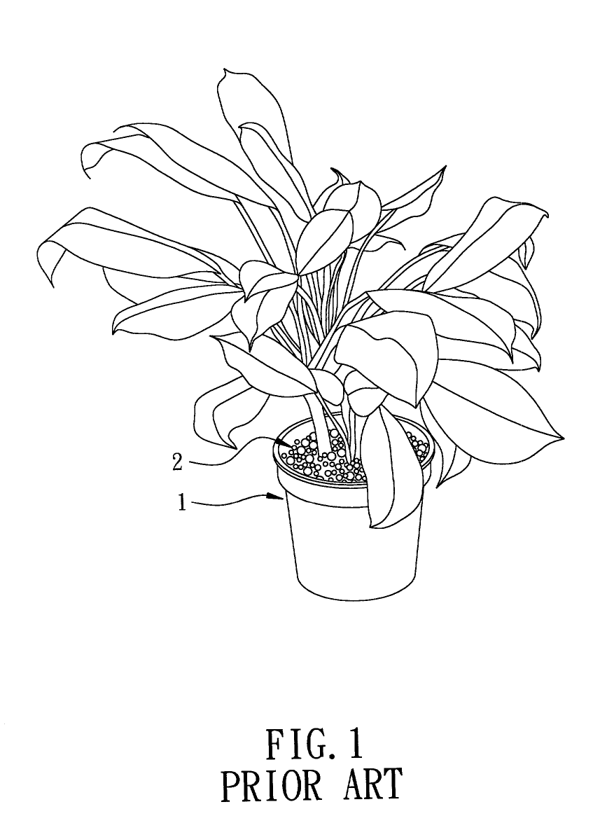

FIG. 2 is an exploded perspective view illustrating a first preferred embodiment of a fertilizer dispensing device according to this invention;

FIG. 3 is a schematic partly sectional view of the first preferred embodiment shown in FIG. 2;

FIG. 4 illustrates the first preferred embodiment in a state of use;

FIG. 5 is an exploded perspective view illustrating a second preferred embodiment of a fertilizer dispensing device according to this invention; and

FIG. 6 is a schematic partly sectional view of the second preferred embodiment shown in FIG. 5.

### DETAILED DESCRIPTION OF THE PREFERRED EMBODIMENTS

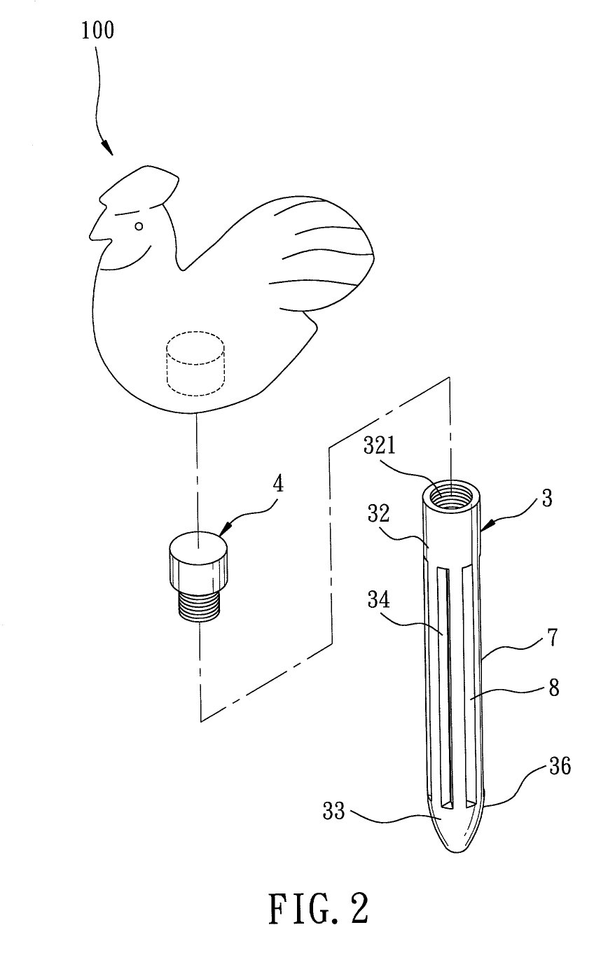

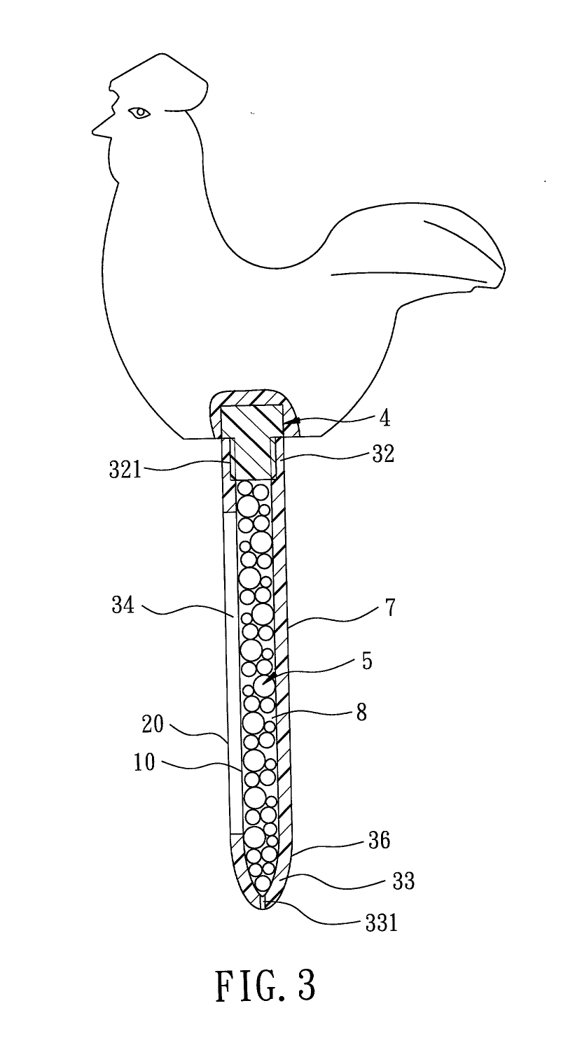

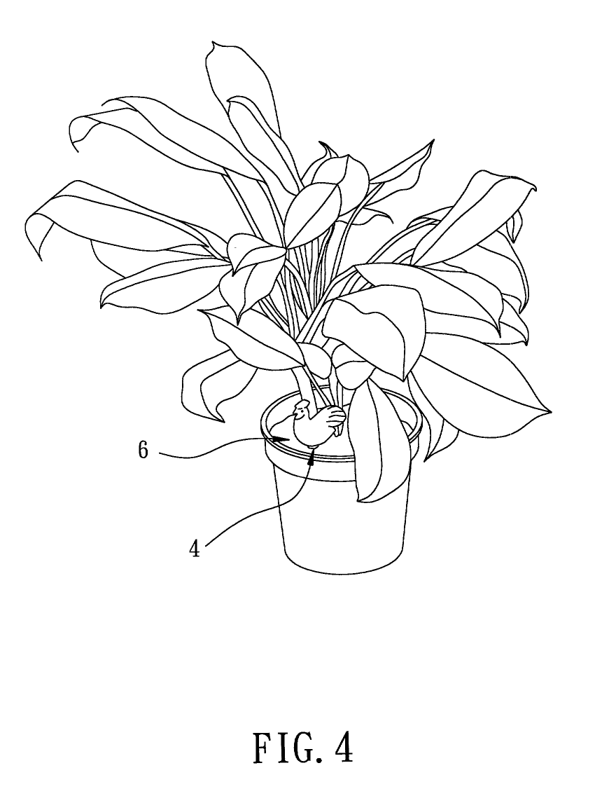

Referring to FIGS. 2, 3, and 4, the first preferred embodiment of the fertilizer dispensing device 100 according to the present invention is shown to have a tubular body 3 adapted to be inserted into and buried by soil 6. The tubular body 3 has a bottom wall 33 with a periphery 36. A tubular wall 7 extends from the periphery 36 of the bottom wall 33 in a longitudinal direction and cooperates with the bottom wall 33 to confine a chamber 8. Preferably, the bottom wall 33 is formed into a conical shape to facilitate insertion of the tubular body 3 into the soil 6. Moreover, the tubular body 3 further has an inner wall surface 10 confronting the chamber 8, and an outer wall surface 20 opposite to the inner wall surface 10 and distal to the chamber 8. The chamber 8 is used to receive granular fertilizers 5, wherein the smallest one of the fertilizers 5 has a first dimension. An upper tubular end 32 extends from the tubular wall 7 in the longitudinal direction to define an access opening 321 to permit filling of the chamber 8 with the granular fertilizers 5. A closure member 4 is disposed to close the access opening 321. Furthermore, a dispensing port 331 is formed in the inner wall surface 10, extends through the outer wall surface 20, and has a second dimension smaller than the first dimension. Thus, the granular fertilizers 5 are retained in the chamber 8 until the granular fertilizers 5 are dissolved so as to be carried through the port 331 and into the soil. Preferably, the dispensing port 331 includes a plurality of slits 34 formed in the tubular wall 7 and angularly displaced from one another. Each of the slits 34 extends in the longitudinal direction.

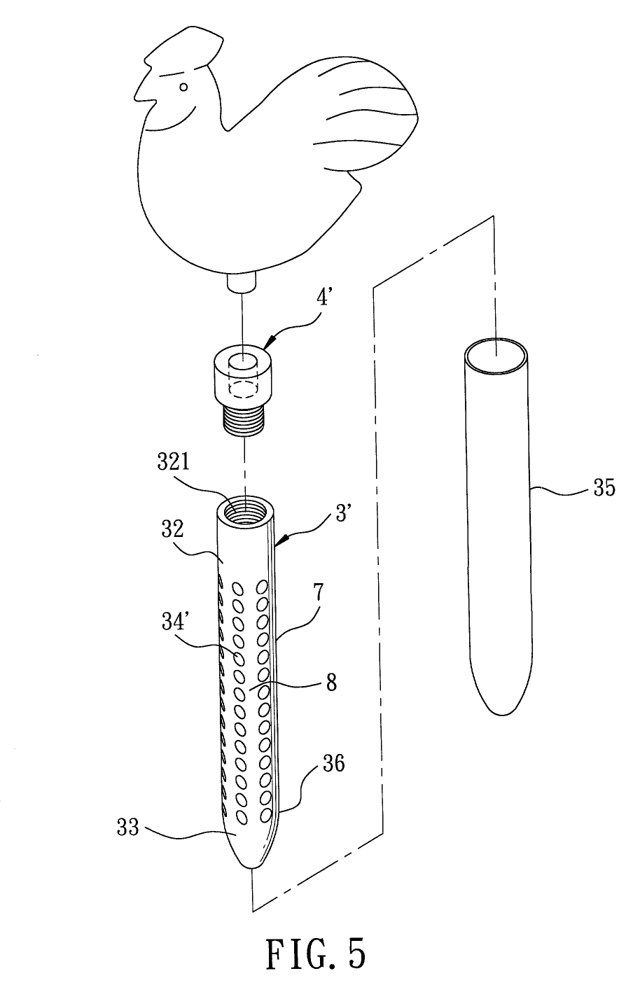

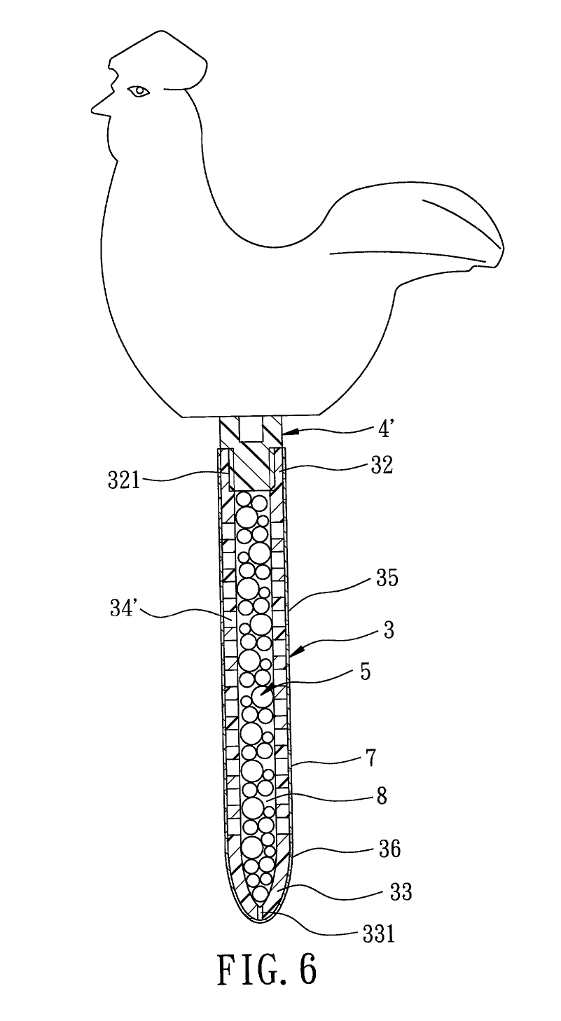

Referring to FIGS. 5 and 6, which illustrate a second preferred embodiment of the present invention, a liquid permeable thin film 35 is wrapped around the tubular body 3. The film 35 is adapted to be inserted and buried by soil together with the tubular body 3. The granular fertilizers 5, when dissolved, are able to penetrate through the liquid permeable thin film 35 and seep into the soil evenly.

In summary, the following are some of the advantages of the fertilizer dispensing device 100 according to the present invention. First, the tubular body 3 of the fertilizer dispensing device 100 is adapted to be inserted into and buried by soil. Thus, the odor of the granular fertilizers 5 can be contained to avoid attracting insects. Second, since the granular fertilizers 5 are buried by soil, the fertilizers 5 can be dissolved by liquid while watering the plants and then be carried into the soil. Third, the tubular body 3 of the fertilizer dispensing device 100 can be wrapped with the liquid permeable thin film 35 to prevent the granular fertilizers 5, the sizes of which are being reduced by the liquid, from exiting the dispensing port 331. In addition, the presence of the liquid permeable thin film 35 allows even distribution of nutrients into the soil and uniform absorption by the roots of plants. Last but not least, the granular fertilizers 5 can be easily refilled through the access opening 321.

While the present invention has been described in connection with what is considered the most practical and preferred embodiments, it is understood that this invention is not limited to the disclosed embodiments but is intended to cover various arrangements included within the spirit and scope of the broadest interpretations and equivalent arrangements.

I claim:

1. A fertilizer dispensing device comprising:
a tubular body adapted to be inserted into and buried by soil, said tubular body having a bottom wall with a periphery, and a tubular wall extending from said periphery of said bottom wall in a longitudinal direction and cooperating with said bottom wall to confine a chamber, said tubular body further having an inner wall surface confronting said chamber, and an outer wall surface opposite to said inner wall surface and distal to said chamber;
granular fertilizers, a smallest one of which has a first dimension;
an upper tubular end extending from said tubular wall in the longitudinal direction to define an access opening to permit filling of said chamber with said granular fertilizers;
a closure member disposed to close said access opening; and
a dispensing port formed in said inner wall surface, extending through said outer wall surface, and having a second dimension smaller than the first dimension, whereby said granular fertilizers are retained in said chamber until said granular fertilizers are dissolved so as to be carried through said port and into the soil.

2. A fertilizer dispensing device according to claim 1, wherein said dispensing port includes a plurality of slits formed in said tubular wall and angularly displaced from one another, each of said slits extending in the longitudinal direction.

3. A fertilizer dispensing device according to claim 1, further comprising a liquid permeable thin film wrapped around said tubular body and adapted to be inserted into the soil together with said tubular body.

4. A fertilizer dispensing device according to claim 1, wherein said bottom wall is formed into a conical shape to facilitate insertion of said tubular body into the soil.
